(19) United States
(12) Patent Application Publication
(10) Pub. No.: US 2021/0393140 A1
(43) Pub. Date: Dec. 23, 2021

(54) TRANSPARENT FACE MASK WITH OPTIONAL TEMPERATURE MONITORING DEVICE AND OPTIONAL DETACHABLE EYE PROTECTION

(71) Applicant: Claritymask, Inc., Bay Shore, NY (US)

(72) Inventor: Gary Rein, New York, NY (US)

(73) Assignee: Claritymask, Inc., Bay Shore, NY (US)

(21) Appl. No.: 17/307,562

(22) Filed: May 4, 2021

Related U.S. Application Data
(60) Provisional application No. 63/019,644, filed on May 4, 2020.

(51) Int. Cl. A61B 5/01; A41D 13/11; A61B 5/00; A61F 9/02; A62B 23/02; A62B 18/08

(52) U.S. Cl. CPC A61B 5/015; A41D 13/1184; A61B 5/7445; A61B 5/6803; A61F 9/026; A62B 23/025; A62B 18/084; A61F 9/029

(57) ABSTRACT

A clear protective mask/face-shield assembly with collapsible temples, which protects the wearer from contaminants and airborne particles, and optionally reads out the wearer's body temperature to the observer through the use of an integral LC flexible forehead thermometer. The assembly includes a rigid single piece clear face covering which protects the user from airborne droplets, and optionally filters incoming, and exhaled air through a filtered port on each side of the mask. The ports also permit air circulation for the comfort of the wearer and to minimize fogging of the mask/shield. An optional safety eye lens protection component can also be added to the face mask.

This application claims priority to U.S. Provisional Patent Application No. 63/019,644 filed May 4, 2020, entitled HINGED TEMPLE TRANSPARENT FACE MASK WITH TEMPERATURE MONITORING DEVICE AND DETACHABLE EYE PROTECTION and is hereby incorporated by reference in its entirety.

### BACKGROUND OF THE INVENTION

The present invention relates to a face mask assembly and more particularly relates to a type of face mask assembly that may be worn by Medical and Healthcare professionals, Industrial workers, and the general public, to minimize the possibility of cross-contamination in hospitals, manufacturing plants, retail spaces, and the general public.

Various types of protective masks and face shields can be found in the prior art, however, current embodiments incorporate several critical shortcomings in attempting to protect healthcare professionals and the general public from airborne droplets and dust particles. Some of these shortcomings are as follows:
Difficulty, discomfort, or inconvenience of attaching similar N95, surgical masks that utilize elastic bands to attach to the ears, or wrap around the head which cause rashes, and abrasion during use over time.
Total visual obstruction of the face in the majority of opaque face masks, limiting casual and visual communication.

It is an object of the present invention to provide an improved protective face mask assembly which utilizes a pivot-hinged temple component to enable the mask to be easily put on, and off without needing to remove any existing headgear, or reach around to the back of the head to affix. Once in position, the contoured planar configuration directs exhaled air towards the sides of the mask, and into an optional HEPA filter ports to minimize fogging.

It is a further object of the present invention to provide, as an option, a protective face mask assembly that incorporates the ability to also visually convey any fluctuation in the wearer's body temperature to an outside observer. Given current conditions, the ability to distinguish any temperature anomalies in a worker, or Healthcare professional's body temperature would be a significant preventative measure to control the spread of any infectious diseases.

Another object of the present invention is to provide a protective face mask assembly which potentially can be worn comfortably by the general public, in leisure situations, and in outdoor scenarios. The current invention incorporates a nose bridge “T” section that can be used in conjunction with a Snap-On eyewear appliance which potentially completes the clear face mask covering to the entire face. In outdoor conditions, the interchangeable eyewear component can be tinted, or incorporate polarizing lenses to block out the sun.

In another optional embodiment, an eye protection visor with hinged temples is integrated to create a “former” which allows a decorated, or branded clear flat fabricated plastic panel to be formed around the fixed molded upper body, and thereby allow the flat clear panel to be changed out when scuffed or abraded.

### SUMMARY OF THE INVENTION

The present invention is directed to a clear protective mask/face-shield assembly with collapsible temples, which protects the wearer from contaminants and airborne particles, and, as an option, simultaneously continually reads out the wearer's body temperature to the observer through the use of an integral Liquid Crystal (LC) flexible forehead thermometer. The assembly includes a rigid single piece clear face covering which protects the user from airborne droplets, and filters incoming, and exhaled air through an optional filtered port on each side of the mask. The ports also permit air circulation for the comfort of the wearer and to minimize fogging of the mask/shield. An optional air-inhibiting flexible silicone-like gasket seal is attached around the periphery of the clear mask portion to enhance comfort, and fit.

The assembly attaches onto the face with two pin-hinged rigid temple pieces that wrap around the user's ears to hold the mask in position, and flexes to create a universal fit for variable ergometric sizing around the ears. Each temple has an integral headed stud post that projects perpendicularly from the side of the temple pieces, and provides a means of attachment for the flexible, interchangeable LC forehead thermometer. The flexible thermometer has variable hole positions, which correspond to the dual post projections on the temple extension to allow the user to adjust the flexible thermometer to be held in compression to the forehead and enables the LC crystals to react with the user's body temperature. In an alternative embodiment the thermometer is affixed to one of the temples on one side and has an expandable flexible band that loops over to the other temple projection to insure ease of attachment and compression on the forehead. The thermometer continuously reads out the user's temperature to the public with a color-coded and numeric read-out. On the bottom of the fixed mask component is a secondary optional means of attachment, which allows an interchangeable fabric, or other “bandana” to be slid in for additional protection below the chin and covering the neck.

On the nose bridge of the clear face mask, is an optional integral molded “T” section that provides a method of attachment for a clear, or tinted eye protection lens visor assembly to be snapped onto the main mask assembly and cover all of the front orifices of the face. The eye cover attachment can be either clear, or tinted, when the mask is used in exterior outdoor locations, creating protective sunglasses for use in public. The extreme edges of the main clear mask incorporate optional gasket(s) that cushion the mask faceplate, and enhance comfort for the user, as well as reducing potential incoming droplets, or particles to enter the mask.

### DESCRIPTION OF THE DRAWINGS

FIG. 1 is a perspective view of the clear main mask assembly of the present invention;

FIG. 1a is a side elevation of the present invention showing the invention on the head of a wearer;

FIG. 2 is a perspective view of the invention.

FIG. 2a is a perspective view of the invention with the temples collapsed, in the closed position;

FIG. 3 is a top view, front and side elevational view of the mask assembly of the present invention.

FIG. 3a is a side elevational view showing molded mask assembly and means of attachment.

FIG. 4 is a perspective view of an alternative embodiment of the mask assembly with the flexible LC thermometer on the wearer;

FIG. 4a is a side elevation of the flexible removable LC thermometer attachment;

FIG. 5 is a perspective view of an alternate embodiment of the mask assembly showing the additional eye protection component.

FIG. 5a is a side elevation of the removable eye protection attachment.

FIG. 6 is a perspective view of an alternative embodiment/construction of the invention showing a clear fabricated panel attached onto a molded clear eyewear/mask assembly.

FIG. 6a is an elevation of the alternative embodiment

FIG. 6b is a perspective of the alternative embodiment showing attachment of a clear fabricated panel attached onto the molded hinged temple assembly

### DETAILED DESCRIPTION OF THE INVENTION

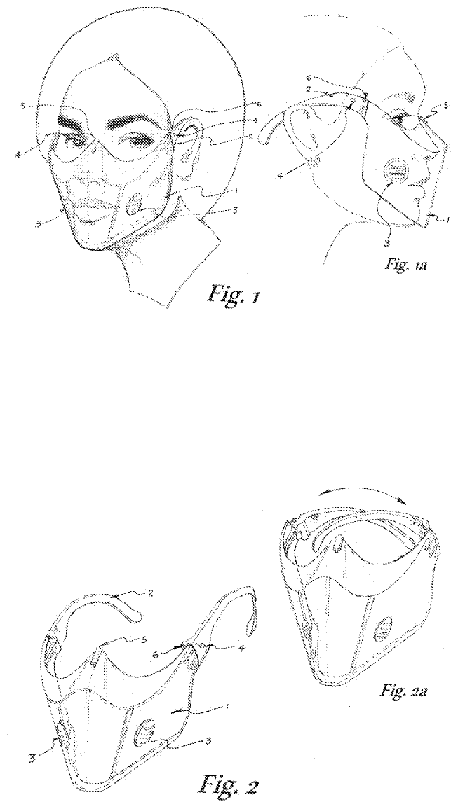

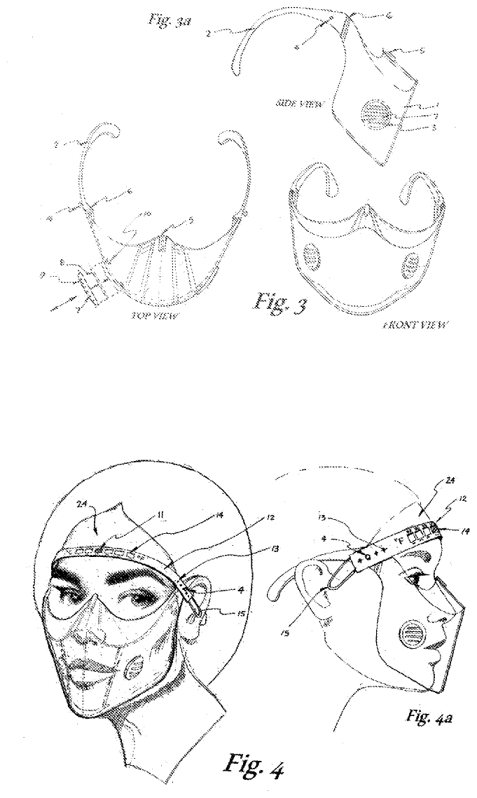

Referring now to FIGS. 1, 1a, 2 and 2a the mask assembly of the present invention is comprised of a clear mask component 1, with pin-hinged 6 collapsible temples 2, which when in the open position create a close-fitting mask. On each side of the main body 1, are optional air filtration ports 3, left and right of the mask body. As shown in FIG. 3 each individual air filtration port 3 has an inner recessed section 10, a die cut HEPA filtration pad 8 and a cover 9 with venting slots 7, to allow air to be inhaled and exhaled to prevent fogging of the clear mask portion 1. The integral recessed port inner portion 10 is part of the main body 1, and allows the filtration pad 8 to move perpendicularly from the resting position to allow air passage to the mask 1. On the two pin-hinged 6 temples 2, are integral headed stud projections 4, that provide an optional means of attaching the flexible LC thermometer strip 12 to the left and right side of the mask 1. Also integral to the main body of the clear mask 1, is a “T” shaped track 5, that provides an optional means for attaching a secondary eye protection visor 16.

Referring now to FIG. 4. There is shown a perspective view of the assembled invention with the flexible LC thermometer 12 mounted onto the hinged temples 2, with the integral side headed stud projections 4, and depicts the LC heat-activated liquid crystal assembly 14 with sur-printed graphics 12 to enable the observer to note any changes in the body temperature of the wearer. The thermometer has two alternative means of attachment; a series of die-cut holes perforated at the ends of the thermometer body 12, that are press-fit over the studs 4 to create appropriate tension to allow the LC crystals 14 to react with forehead of the wearer 24. In an alternative attachment method, to create variable tension, the thermometer body 12 has one, or two ends with a flexible elastic band 15 that maintains continual pressure along the forehead 24. When activated the color-coded optimal body temperature thermometer will turn green.

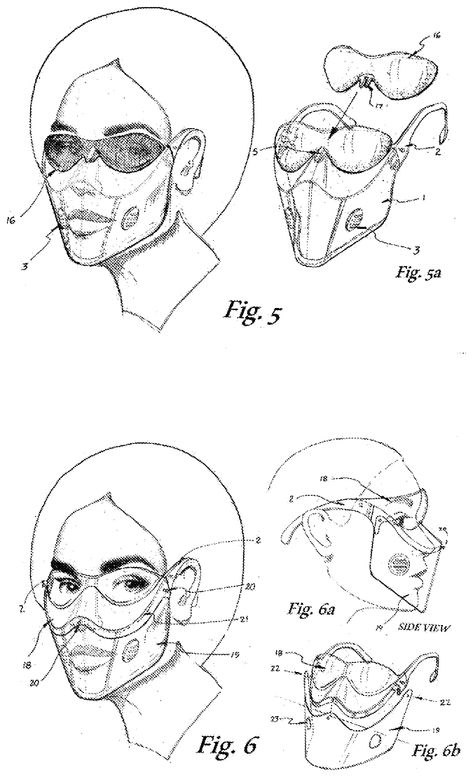

Referring now to FIG. 5. There is shown the clear mask assembly 1 in perspective in an alternative embodiment, where an additional eye protective visor 16 (tinted, or non-tinted) is removably attached to the main clear mask 1, press-fitting to the main mask's 1 nose bridge “T” track 5. FIG. 5a is an exploded perspective of the full mask assembly 1, with the eye protection visor 16 attaching to the nose bridge “T” track 5.

Referring now to FIG. 6. There is shown an alternative mask construction embodiment shown in perspective on the user. In this form, the face mask is comprised of; an upper eyewear visor 18, with lateral molded hinged-temples 2, and a fabricated clear visor panel 19 that conforms to the periphery of the molded upper portion 18. The upper eyewear visor 18 has an integral fixed locator track 21 on the extreme vertical perimeter lip of the front of the mask, that in conjunction with a series of headed stud projections 20, provides for alignment of the lower clear fabricated visor 19. A matching series of alignment holes 22 are on the clear lower visor 19. Lateral holes 23 in the lower visor 19 enable the attachment of the air filtration port assemblies 3 on the left and right sides of the visor 19.

Although a specific embodiment of the invention has been disclosed herein it is to be understood that various modifications can be made to the described embodiment without departing from the scope of the claimed invention, which modification would be apparent to one skilled in this art area.

1. A protective face mask assembly to be worn on the head of the wearer comprising:
(a) a substantially transparent plastic face mask that covers the nose and mouth of the user and attaching to the head using left and right hinged folding temples that attach behind the ears to hold the assembly in place,
(b) stud projections on said hinged folding temples at opposite locations to enable the attachment of secondary elements,
(c) an integral “T” section projection on a nose bridge of said face mask to provide ancillary attachments, and
(d) a protruding perimeter lip around the periphery of said face mask to reduce abrasion to the wearer.

2. A protective face mask assembly in accordance with claim 1 further including a press-fit attachment portion around the periphery of the face mask to press-fit a silicone gasket to reduce air intrusion.

3. A protective face mask assembly in accordance with claim 1 further including a flexible thermometer to provide a continuous visual display read-out of the wearer's body temperature said flexible thermometer comprising:
(a) a series of perforations at each end of the thermometer, and
(b) an elastic band on each end of the thermometer to attach removably to the stud projections;
wherein
(c) said thermometer has heat sensitive liquid crystals which emanate a variety of colors at different temperature wave lengths; and
(d) individual temperatures are displayed in self-contained compartments, showing individual colors, and respective graphic temperatures visible to the public.

4. A protective mask assembly in accordance with claim 1 further including a safety eye lens protection component to be added as desired to the face mask wherein:
(a) said safety eye lens protection component is removably attached to said nose-bridge of said face mask,
(b) said safety eye lens protection component press-fits onto said “T” nose-bridge, and
(c) said safety eye lens component is contoured to the exact configuration of an upper periphery of said face mask, thereby eliminating any potential apertures and unitizing said safety eye lens component to said face mask to minimize the possibility of airborne droplets entering into said face mask.

5. A protective face mask assembly in accordance with claim 1 further including a single, or double air filtering port included in an integral pocket in said face mask, having a die-cut HEPA multi-layered filtration pad; and further including a snap-in molded removable separate cover that allows for filter pad replacement.

6. The protective face mask in accordance with claim 3 wherein said elastic band comprises spaced-apart resilient collapsible hinged temple sections interconnected by a curved forward section and wherein terminal ends of said temple sections have rearwardly extending tabs.

7. A protective face mask assembly for medical, industrial and dental personnel, or the general public comprising:
(a) a clear mask having head-engaging hinged folding temples;
(b) said temples having perpendicular stud protrusions to optionally attach a flexible thermometer; and
(c) an integral “T” section on an extreme upper nose bridge of said face mask to attach an additional contoured eye safety element for full eye, nose, and mouth coverage.
